(19) United States
(12) Patent Application Publication
(10) Pub. No.: US 2012/0171309 A1
(43) Pub. Date: Jul. 5, 2012

(54) FORMULATIONS AND METHODS FOR INCREASING EFFICACIES AND IMPROVING FLAVORS OF FORMULATIONS CONTAINING GYNOSTEMMA PLANT

(76) Inventors: Harunobu Amagase, Phoenix, AZ (US); Richard Handel, Little Falls, NJ (US)

(21) Appl. No.: 13/394,643

(22) PCT Filed: Sep. 8, 2009
(86) PCT No.: PCT/US09/56234

§ 371 Date: Mar. 7, 2012

(51) Int. Cl. A61K 36/00; A61P 43/00; A61P 39/06; A61K 31/704; A61P 3/00

(52) U.S. Cl. 424/725; 514/26

(57) ABSTRACT

Formulations and methods for increasing efficacies and flavors of jiaogulan (Gynostemma pentaphyllum and related species) by combining materials derived from whole plant with other herbal and nutritional materials for the purpose of improving general well-being in mammals by increasing metabolic rate through fatigue-reducing, tonic, antioxidant and adaptogenic effects of the invention.

### FIELD OF THE INVENTION

The invention relates generally to formulations and methods for increasing efficacies and flavors of formulations containing Gynostemma plant, either alone or in combination with other herbal and/or nutritional ingredients, for the purpose of improving general well-being in mammals by increasing metabolic rate through fatigue-reducing, tonic, antioxidant and adaptogenic effects of the invention.

### BACKGROUND OF THE INVENTION

Gynostemma pentaphyllum (jiaogulan) is most often consumed as an herbal tea prepared from dried leaves, and is also available as an alcohol extract and in capsule or pill form. However, it has never been used as a combination of whole plant material. Jiaogulan is known as an adaptogen and antioxidant, and it has been shown in laboratory, animal and human studies to increase immune function and to inhibit carcinogenesis and tumor growth. Due to its adaptogenic effects, it is frequently referred to as “Southern Ginseng,” although as a member of family Cucurbitaceae, it is not botanically related to the true Panax ginsengs of the Araliaceae family. Its adaptogenic constituents include dammarane (triterpenoid) saponins identical to the ginsenosides found in the well-known medicinal plant Panax ginseng, as well as a complement of more than 100 gypenosides, a series of unique dammarane saponins that, although structurally related to ginsenosides, occur only in Gynostemma species. Jiaogulan and its gypenosides have been shown to lower cholesterol and blood lipid levels. The plant is best known for its use as an herbal medicine in traditional Chinese medicine. Until recently it was a locally known herb used primarily in regions of southern China. It is described by the local inhabitants as the “immortality herb”, because people within the Guizhou Province, where jiaogulan is consumed regularly, have a history of living to a very old age.

Jiaogulan is nontoxic in normal doses, produces a non-specific defensive response to stress, and has a normalizing influence on the body. Adaptogens such as jiaogulan normalize the hypothalamic-pituitary-adrenal axis, and as defined, adaptogens constitute a new class of natural, homeostatic metabolic regulators. However, they are also functional at the level of allostasis which is a more dynamic reaction to long term stress, lacking the fixed reference points of homeostasis.

Needless to say, stress is the major cause of uncomfortable physiological and psychological symptoms and it is implicated as having a major role in the etiology and progression of cardiovascular disease, diabetes mellitus, stroke, obesity and other serious diseases and conditions. Reducing stress is critical to maintaining a healthy life and to reducing the risks of these stress-related conditions. Therefore, effective, safe and convenient adaptogens have become popular as a non-invasive means of preventing the harmful effects of physiological, emotional and mental stress. Low metabolic rate is strongly related to mood, stress and other psychological symptoms, and also is a strong risk factor for heart attacks, strokes, diabetes and other diseases. Based on new research, increased metabolic rate or thermogenesis is connected to weight loss, but it also influences psychological symptoms. While many people are advised by their physicians and health practitioners to prevent or counteract the effects of stress by adopting relaxation techniques or by obtaining psychological treatments, it is often difficult to change one's lifestyle and as a result, the incidence of stress-related diseases is rising sharply.

As jiaogulan is neither well known nor frequently consumed in the western world, a powerful and easy-to-take jiaogulan preparation is needed. Accordingly, a need exists for increasing efficacies and flavors of preparations containing jiaogulan, either alone or in combination with other herbal and/or nutritional ingredients, for the purpose of improving general well-being in mammals by increasing metabolic rate through fatigue-reducing, tonic, antioxidant and adaptogenic effects of the invention, thereby avoiding invasive and rigorous medical intervention.

### SUMMARY OF THE INVENTION

Here, the inventors demonstrate versatile formulations and methods for increasing efficacies and flavors of preparations containing jiaogulan in combination with other herbal and nutritional ingredients for the purpose of improving general well-being in mammals by increasing metabolic rate through fatigue-reducing, tonic, antioxidant and adaptogenic effects of the invention.

The jiaogulan blend in this invention is a formulation of whole plant high-saponin water extract, freeze-dried whole herb and juice from the leaf, stem and root, but it can be any combination of any type of material derived from Gynostemma pentaphyllum and related species. Furthermore, it can be combined with any other materials, such as herbs, vitamins and others. The following are the examples of the materials, but this invention does not limit to these: Preparations of goji (Lycium barbarum), astragalus (Astragalus membranaceus), codonopsis (Codonopsis pilosula), jujube (Zizyphus jujuba), longan (Dimocarpus longan), luohanguo (Siraitia grosvenorii), rhodiola (Rhodiola rosea), schisandra (Schisandra chinensis), açai (Euterpe oleracea), amalaki (Emblica aronia (Aronia melanocarpa), camu-camu (Myrciara dubia), elderberry (Sambucus nigra), ginger (Zingiber officinale), Japanese sophora (Sophora japonica), blueberry (Vaccinium angustifolium), bilberry (Vaccinium myrtillis), lychee (Litchi chinensis), cranberry (Vaccinium macrocarpon), cherry (Prunus cerasus) and reishi mushroon preparations.

Accordingly, the present invention provides a method for increasing efficacies and flavors of preparations containing jiaogulan in combination with other herbal and nutritional ingredients for the purpose of improving general well-being in mammals by increasing metabolic rate through fatigue-reducing, tonic, antioxidant and adaptogenic effects of the invention, the method comprising of administering a composition consisting of jiaogulan whole plant high-saponin water extract, freeze-dried whole jiaogulan herb and juice from jiaogulan leaf, stem and root, with preparations of goji, astragalus, codonopsis, jujube, longan, luohanguo, rhodiola, schisandra, açai, amalaki, aronia, camu-camu, elderberry, ginger, Japanese sophora, blueberry, bilberry, lychee, cranberry, cherry and reishi mushroon preparations, whereby the subject's health benefits are increased. In an alternate preferred embodiment, the composition comprises a juice formula.

In another embodiment, the invention provides a method of improving the flavor of the formulations/preparations by oral administration of the additional nutritional ingredients to a subject, the method comprising administering a composition comprising whole Gynostemma plant, such as jiaogulan high-saponin water extract, freeze-dried whole jiaogulan herb and juice from jiaogulan leaf, stem and root, with preparations of goji, astragalus, codonopsis, jujube, longan, luohanguo, rhodiola, schisandra, açai, amalaki, aroma, camu-camu, elderberry, ginger, Japanese sophora, blueberry, bilberry, lychee, cranberry and cherry, whereby the subject's total general well-being is increased by fatigue-reducing, tonic, antioxidant and adaptogenic effects of the invention. In an alternate preferred embodiment, the composition comprises whole jiaogulan plant and concentrated active compounds (gypenoside saponins, polysaccharides and others) of jiaogulan.

In yet another embodiment, the invention provides a food formulation consisting of an effective amount of jiaogulan and/or preparations of goji, astragalus, codonopsis, jujube, longan, luohanguo, rhodiola, schisandra, açai, amalaki, aronia, camu-camu, elderberry, ginger, Japanese sophora, blueberry, bilberry, lychee, cranberry, cherry, and reishi mushroon preparations and at least one other food ingredient. In an alternate preferred embodiment, the composition comprises a juice formula.

The invention also encompasses the use of jiaogulan and/or preparations of goji, astragalus, codonopsis, jujube, longan, luohanguo, rhodiola, schisandra, açai, amalaki, aronia, camu-camu, elderberry, ginger, Japanese sophora, blueberry, bilberry, lychee, cranberry, cherry, and reishi mushroon preparations for the manufacture of food formulations to improve total general well-being by fatigue-reducing, tonic, antioxidant and adaptogen effects in a subject ingesting the food formulation. Also within the invention are jiaogulan preparations, preferably jiaogulan juice, packaged and presented for use in the improvement of various health aspects, and to increase total general well-being by fatigue-reducing, tonic, antioxidant and adaptogenic effects of the invention.

Other objects, features and advantages of the present invention will become apparent after review of the specification, claims and drawings.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed non-provisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

The term “administering” shall refer to providing the claimed formulation for oral consumption. In certain embodiments, the method of administering the claimed formulation shall include the steps of fasting for 12 hours; orally consuming the claimed formulation; and subsequently consuming a nutritional beverage.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

While commercially available Gynostemma plant, such as jiaogulan dried leaf and its extracts have been known to have various health benefits, we have newly discovered increased efficacies of health benefits from a combination of factors derived from the whole plant (high-gypenoside saponin plant extract, freeze-dried whole herb and juice from leaf, stem and root), hereinafter referred to as jiaogulan blend.

We furthermore formulated the aforementioned jiaogulan blend with various other herbal and nutritional materials as the new formulation, which we found to have unexpectedly much better effects than jiaogulan leaf extract or the jiaogulan blend alone.

It is difficult or impractical to take sufficiently high amounts of the various ingredients of the formulation at one time in solid dosage forms such as tablets or capsules. A liquid dosage form can accommodate an effective level of ingredients in a conveniently small serving size. The health effects of jiaogulan are best realized when it is consumed on a daily basis, yet the taste of jiaogulan is not appealing to the majority of people, which is a deterring factor to compliance. The primary biologically active principles of Gynostemma plant are dammarane saponins (gypenosides), the taste of which are intensely bitter and unpalatable. This makes it difficult to formulate a high-saponin Gynostemma formulation in a liquid form without adding flavors or taste-masking materials. In this invention, we have developed a formulation that contains a unique combination of ingredients in a convenient liquid dosage form with a palatable and consumer-acceptable flavor system.

Herein, the inventors provide formulations and methods for increasing efficacies and flavor of Gynostemma pentaphyllum and related species (jiaogulan) by combining different preparations of jiaogulan extract, juice and freeze-dried materials with other herbal and nutritional materials for the purpose of improving general well-being in mammals by increasing metabolic rate through fatigue-reducing, tonic, antioxidant and adaptogenic effects of the invention.

When we combined jiaogulan leaf extract with jiaogulan juice and its freeze-dried materials, metabolic rate was increased more than with jiaogulan leaf extract itself.

Furthermore, the formulation containing jiaogulan extract, juice and freeze-dried materials with other herbal and nutritional materials is unexpectedly much better in increasing metabolic rate than are either jiaogulan leaf extract alone or a blend of jiaogulan extract, juice and freeze-dried materials without the addition of other herbal and nutritional materials. The formulation has a significant effect at 2 hours after intake on resting metabolic rate and is expected to last longer than 2 hours. On the other hand, jiaogulan leaf extract only has no effect at 2 hours after intake. Thus, this new formula has unexpectedly much better effect on metabolic rate than jiaogulan leaf extract itself.

The jiaogulan plants in this invention are the plants belonging to the genus Gynostemma, such as Gynostemma pentaphyllum (synonyms Gynostemma pedatum, Vitis pentaphylla and others). Other species of Gynostemma are within the invention, and they include G. aggregatum, G. burmanicum, G. cardiospermum, G. compressum, G. guangxienense, G. laxiflorum, G. laxum, G. longipes, G. microspermum, G. pallidinerve, G. pentagynum, G. pubescens, G. simplicifolium, G. yixingense and others. All are referred to by the Chinese pinyin term jiaogulan, meaning “twisting vine orchid.” Gynostemma is an herbaceous vine of the family Cucurbitaceae (cucumber or gourd family). The most suitable plant for this invention is Gynostemma pentaphyllum (known as jiaogulan, Amachazuru, Dungkulcha, Fairy Herb, Gynostemma, Immortality Herb, Jiao Gu Lan, Miracle Grass, Penta Tea, Southern Ginseng, Xian Cao, Xianxao, five-leaf ginseng, or poor man's ginseng). The preferred portion for this invention is the leaf, stem and root of this plant. The other parts of the plant may also be utilized for this invention. These materials can be processed as juice, dried or freeze-dried by processing and/or extraction methods commonly known in the art.

In addition, the materials derived from the cell culture of the plants can also be utilized as materials for this invention. The juice or extracts of jiaogulan plants in this invention are preferably the preparations made from plant materials prepared or extracted with water, alcohol, hydroalcoholic menstruums or supercritical fluid carbon dioxide. For preparation or extraction, the Gynostemma plants can be squeezed or crushed with or without a moderate temperature to effect extraction efficiency, as is routinely understood in the art. It is also possible to crush and homogenize the plants to make the juice after separating leaf, stem, root and other parts. The extract prepared for dietary intake can be utilized as is, in concentrated fluid or powder form after concentration under vacuum or by lyophilization.

Furthermore, the jiaogulan materials can be mixed with purified or synthesized saponins, such as gypenoside saponins or any other suitable saponins, such as the ones derived from ginseng, garlic or other herbs. These materials are not limited to the extract from water, but also from any appropriate solvents.

The administrative dosage of the preparation effective to increasing efficacies and flavors of jiaogulan by combining herbal and nutritional materials on total general well-being by fatigue-reducing, tonic, antioxidant and adaptogenic effects of the invention through the various mechanisms we incorporated in this invention depends on the ingredients as well as the age, body weight or body condition of the subject. It is recommended to take orally 0.001 oz (0.03 ml) through 33 oz (990 ml) in a day by adults as a liquid preparation of the combination of whole jiaogulan plant high-saponin water extract, freeze-dried whole jiaogulan and juice from jiaogulan leaf, stem and root with preparations of goji, astragalus, codonopsis, jujube, longan, luohanguo, rhodiola, schisandra, açai, amalaki, aronia, camu-camu, elderberry, ginger, Japanese sophora bud, blueberry, bilberry, lychee, cranberry and and/or reishi mushroon preparations. The more feasible and preferred dosage ranges approximately from 1 oz (30 ml) to 8 oz (240 ml) of the preparation per day for an adult human.

The range for each ingredients may be following, but not limited to these:
Gynostemma plant preparations: 0.001 microgram to 100,000 g per day. Any sources are applicable for this invention, including whole plant, leaf, stem, root or any other parts of the plant. Furthermore, any kinds of extraction can be used for this preparation, such as water (cold, warm or hot), allowable solvents alone or combination in any ratios.
Goji: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Astragalus: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Codonopsis: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Jujube: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Longan: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Luohanguo: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Rhodiola: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Schisandra: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Açai: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Amalaki: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Aronia: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Camu-camu: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Elderberry: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Ginger: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Japanese sophora: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Blueberry: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Bilberry: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Lychee: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Cranberry: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Cherry: 0.001 microgram to 100,000 g per day. Any parts of the plant can be blended together and applicable for this invention. Furthermore, any kinds of extraction can be used for this preparation, such as water (cold, warm or hot), allowable solvents alone or combination in any ratios.
Reishi mushroon: 0.001 micro g to 100,000 g per day. Any parts of the plant can be blended together and applicable for this invention. Furthermore, any kinds of extraction can be used for this preparation, such as water (cold, warm or hot), allowable solvents alone or combination in any ratios.

In addition, any additional flavoring materials can be mixed together to improve palatability of the above combinations by masking the bitterness or unfavorable flavors. For such purposes, ordinary flavoring agents may be used, but preferably, cane juice, cherry juice and/or any other berry flavors can be added. Flavors are very important for continuous daily intake of these formulations to maintain healthy conditions and life style.

A preferred formulation according to this invention is provided below:

| | Weight |
|---|---|
| FORMULA: | ratio (%) |
| Jiaogulan (Gynostemma pentaphyllum) Blend | 29.97% |
| Goji berry (Lycium barbarum) | 17.21% |
| Acai (Euterpe oleracea) | 8.61% |
| Aronia (Aronia melanocarpa) | 1.08% |
| Camu camu (Myrciaria dubia) | 1.08% |
| Elderberry (Sambucus nigra) | 1.08% |
| Jujube (Ziziphus zizyphus) | 0.72% |
| Amalaki (Phyllanthus emblica) | 0.43% |
| Schisandra (Schisandra chinensis) | 0.43% |
| Cranberry (Vaccinium oxycoccos) | 0.43% |
| Luo Han Guo (Siraitia grosvenor;;) | 0.24% |
| Longan (Dimocarpus longan) | 0.22% |
| Pomegranate (Punica granatum) | 0.22% |
| Bilberry (Vaccinium myrtillus) | 0.09% |
| Blueberry (Vaccinium corymbosum) | 0.17% |
| Lychee (Litchi chinensis) | 0.22% |
| cherry (Prunus cerasus) | 0.22% |
| Japanese sophora (Styphnolobium japonicum) | 34.42% |
| Rhodiola (Rhodiola rosea) | 4.30% |
| Codonopsis (Codonopsis pilosula) | 0.54% |
| Astragalus (Astragalus membranaceus) | 0.36% |
| Ginger (Zingiber officinale) | 0.22% |

The following examples describing materials and methodology are offered for illustrative purposes only, and are not intended to limit the scope of the present invention.

### III. Examples

### Example

### A Controlled Human Clinical Study.

This example describes a human clinical study which examined the efficacy of orally consumed formulation, which contains a jiaogulan blend from whole plant high-saponin extract, freeze-dried whole herb and juice from leaf, stem and root; goji juice from fresh Lycium barbarum fruit; standardized extracts of astragalus root, codonopsis root, jujube fruit, longan fruit, luo han guo fruit, Tibetan rhodiola root, schisandra fruit, freeze-dried açai berry, amalaki fruit, aronia berry, camu camu fruit, elderberry fruit, ginger root, Japanese sophora bud, wild blueberry fruit, bilberry fruit, lychee fruit, and cherry fruit, compared to jiaogulan leaf extract (A) alone and to a jiaogulan blend (B) on resting metabolic rate (RMR) in healthy human adults after a single bout of consuming 60 ml (2 oz) of each preparation. The formulation was separately consumed immediately before a nutritional beverage (360 kcal) after over 12 hours fasting.

TABLE 1

| | RMR change |
|---|---|
| | from baseline |
| Preparations | (kcal) |
| Control | 139 |
| Gynostemma pentapyllum leaf extract alone (A) | 134 |
| Blend of Gynostemma pentapyllum (B)* | 178 |
| Blend of Gynostemma pentapyllum (B) plus various | 231 |
| herbs [claimed formulation] | |

*(B) contains reconstituted blend of Gynostemma pentapyllum from standardized high gypenoside saponin extract, freeze-dried whole herb and juice from leaf.

The effect of a single bout of consuming either Gynostemma pentapyllum leaf extract only (A), a blend of Gynostemma pentapyllum (B) and a blend of Gynostemma pentapyllum (B) plus various herbs [the claimed formulation] on resting metabolic rate (RMR) was investigated by hand-held indirect calorimeter in a human clinical trial (n=10). Each value indicates changes from baseline RMR (1,703 kcal). Table 1 illustrates the results of this study.

The jiaogulan blend (B), which contains reconstituted standardized high gypenoside saponin extract, freeze-dried whole herb and juice from leaf, stem and root increased RMR by 178 kcal over baseline level (1,703 kcal). Furthermore, after consuming the claimed formulation (combining jiaogulan blend (B) plus various herbs and nutritional materials) (n=10) RMR was significantly increased by 231 kcal, which is about 14% of baseline level, higher than the control or jiaogulan leaf extract (A) alone. On the other hand, jiaogulan leaf extract alone (A) did not increase RMR over baseline, and was significantly lower than the effect on RMR after the consumption of the claimed formulation.

These results indicate that the synergistic effect of various herbs and nutritional materials added to the jiaogulan blend (B) in the formulation causes an unexpected significant increase in the effect of jiaogulan as a new discovery.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms thereof as come within the scope of the following claims.

1. A method of increasing efficacies and flavors of formulations containing jiaogulan (Gynostemma pentaphyllum and related species) by combining high gypenoside saponin jiaogulan plant extract, freeze dried whole jiaogulan herb and juice from jiaogulan leaf, stem and root for the purpose of improving general well-being in mammals by reducing fatigue, increasing metabolic rate, and by means of tonic, antioxidant and adaptogenic effects.

2. A method of increasing efficacies and flavors of formulations containing jiaogulan (Gynostemma pentaphyllum and related species) by combining high gypenoside saponin jiaogulan plant extract, freeze dried whole jiaogulan herb and juice from jiaogulan leaf, stem and root with crude, purified or isolated saponins, such as gypenoside saponins or any other suitable saponins, such as the ones derived from ginseng, garlic or other herbs for the purpose of improving general well-being in mammals by reducing fatigue, increasing metabolic rate, and by means of tonic, antioxidant and adaptogenic effects.

3. A method of increasing efficacies and flavors of formulations containing jiaogulan (Gynostemma pentaphyllium and related species) by combining jiaogulan with various herbal and nutritional materials for the purpose of improving general well-being in mammals by reducing fatigue, increasing metabolic rate, and by means of tonic, antioxidant and adaptogenic effects.

4. A food formulation comprising an effective amount of jiaogulan as a blend comprising high gypenoside saponin jiaogulan plant extract, freeze dried whole jiaogulan herb and juice from jiaogulan leaf, stem and root for the purpose of improving general well-being by reducing fatigue, increasing metabolic rate, and by means of tonic, antioxidant and adaptogenic effects in mammals ingesting said food formulation and at least one other food ingredient.

5. A food formulation comprising an effective amount of jiaogulan as a blend comprising high gypenoside saponin jiaogulan plant extract, freeze dried whole jiaogulan herb and juice from jiaogulan leaf, stem and root with crude, purified or isolated saponins, such as gypenoside saponins or any other suitable saponins, such as the ones derived from ginseng, garlic or other herbs for the purpose of improving general well-being by reducing fatigue, increasing metabolic rate, and by means of tonic, antioxidant and adaptogenic effects in mammals ingesting said food formulation and at least one other food ingredient.

6. A food or dietary supplement formulation comprising an effective amount of jiaogulan as a blend comprising high gypenoside saponin jiaogulan plant extract, freeze dried whole jiaogulan herb and juice from jiaogulan leaf, stem and root for the purpose of improving general well-being by reducing fatigue, increasing metabolic rate, and by means of tonic, antioxidant and adaptogenic effects in mammals ingesting said food or dietary supplement formulation and at least one other food or dietary supplement ingredient.

7. (canceled)

8. (canceled)

9. (canceled)

10. (canceled)
